(19) United States
(12) Patent Application Publication
(10) Pub. No.: US 2021/0008271 A1
(43) Pub. Date: Jan. 14, 2021

(54) METHOD AND DEVICE FOR THE EXTRACORPOREAL REMOVAL OF PATHOGENS AND/OR AN EXCESS OF COMPONENTS FROM A CELL SAMPLE OF A PATIENT

(71) Applicant: TOPOSNOMOS LTD., MUNCHEN (DE)

(72) Inventor: Walter Schubert, Leipzig (DE)

(21) Appl. No.: 16/092,402

(22) PCT Filed: Apr. 4, 2017
(86) PCT No.: PCT/EP2017/000419

§ 371 Date: Oct. 9, 2018

(30) Foreign Application Priority Data
Apr. 8, 2016 (DE) 10 2016 106 510.2

(51) Int. Cl. A61M 1/36; C12N 5/09; C12N 5/079; G01N 33/574; G01N 33/68

(52) U.S. Cl. CPC A61M 1/3687; C12N 5/0693; C12N 5/0618; G01N 33/57434; A61M 2202/09; A61M 1/3681; G01N 2800/28; A61M 2202/0415; G01N 33/6896

(57) ABSTRACT

The invention relates to a method for the extracorporeal removal of pathogens and/or an excess of components from a cell sample of a human or animal patient suffering from an illness. The method comprises the steps: a) determination of at least one protein cluster (CMP) that is characteristic of the illness of the patient; b) preparation of the cell sample of the patient; and c) extracorporeal removal of components having the at least one determined protein cluster from the cell sample. The invention further relates to a device for carrying out said method.

### DESCRIPTION

The invention relates to a method and a device for extracorporeal removal of pathogenic and/or an excess of components from a cell sample of a human or animal patient.

The incorporeal treatment of many diseases is often associated with strong side effects or even impossible heretofore. Alternatively, devices are known, which are outside of the body (extracorporeal) of the patient and with the aid of which cell samples of the patient can be treated and subsequently again be returned. Hereby, a more gentle treatment of the concerned disease is possible in some cases, since the immune system of the patient is for example affected to lower extent by the treatment than it would be possible by an incorporeal treatment.

It is to be regarded as disadvantageous that the known extracorporeal treatment methods and the devices used for performing them are comparatively unspecific and therefore are only suitable for correspondingly unspecific treatment of relatively few diseases.

It is the object of the present invention to specify a method and a device for extracorporeal removal of pathogenic and/or an excess of components from a cell sample of a human or animal patient, which allow more specific and flexible treatment of a plurality of diseases.

According to the invention, the objects are solved by a method having the features of claim 1 as well as by a device according to claim 16. Advantageous configurations with convenient developments of the invention are specified in the respective dependent claims, wherein advantageous configurations of the method are to be regarded as advantageous configurations of the device and vice versa.

A first aspect of the invention relates to a method for extracorporeal removal of pathogenic and/or an excess of components from a cell sample of a human or animal patient suffering from a disease, in which at least the steps a) determining at least one protein cluster (CMP), which is characteristic of the disease of the patient, b) providing the cell sample of the patient and c) extracorporeal removal of components, which have the at least one determined CMP, from the cell sample are performed. Hereby, a plurality of diseases can be highly specifically treated with few side effects, since only the affected components of the cell sample are removed outside of the body of the patient, while cells of the cell sample not affected by the disease are not removed or remain at least largely unchanged. In the context of the present invention, the term “removal” does not only include partial or complete “removal” of the concerned components, but alternatively or additionally also partial or complete inactivation or destruction of these components, whereby they are at least no longer present in their pathogenic form in the cell sample or are depleted as greatly as possible.

In the context of the present invention, a combinatorial molecular phenotype (CMP) is understood by a protein cluster, which describes colocalized and/or anti-colocalized CDs (clusters of differentiation), proteins and/or molecules at a certain location of a cell. Accordingly, clusters of CMPs represent regions of colocalized and/or anti-colocalized CDs, proteins or other molecules in a cell or a cell complex of the cell sample. In this context, a CMP can for example be specified in the form of a binary code, wherein it is possible to code via each figure of the binary number (i.e. via each “bit”) if a predetermined CD/protein/molecule occurs in the cell at a certain preset location and/or in a concentration exceeding a certain limit value (e.g. L, 1 or true). In the other case, that is if the predetermined CD/protein/molecule does not occur at this location of the cell or only in a concentration below a limit value, a deviating coding (e.g. A, 0 or false) is used. Similarly, it can be provided that certain CDs/proteins/molecules are only sometimes colocalized with one or more other CDs/proteins/molecules and sometimes not colocalized. These CDs/proteins/molecules can be coded with “wildcards” (e.g. W, *).

The invention is based on the realization that certain CDs/proteins/molecules function as so-called leading proteins in disease-specific and/or patient-specific manner. Proteins are often coupled to each other as clusters or networks (corresponding to the said CMP motifs) at a high level of the molecular cell organization, wherein this coupling is controlled by leading proteins: If the leading proteins are inhibited or removed, these clusters decompose, which results in a loss of their biological function. Therefore, numerous diseases can be effectively treated at least substantially free from side effects by determining the CMP or the group of CMPs, which is or are characteristic of a certain disease, whereupon the concerned components of the cell sample, which carry these pathologic “markers”, are extracorporeally removed from the cell sample or inactivated.

Basically, the method according to the invention can be performed one time or multiple times depending on the disease and the success of treatment. For example, multiple cycles can be performed in certain consistent or varying intervals. The number and frequency of the method passages should be determined and controlled by a physician.

Therein, it has proven advantageous if a body fluid, in particular blood and/or blood plasma, and/or a tissue sample of the patient are provided as the cell sample. Hereby, the cell sample can be optimally selected depending on the disease to be treated.

Further advantages arise by determining the at least one CMP based on a database and/or by means of a multi-epitope ligand cartography (MELK) and ICM method (multi-epitope ligand cartography/imaging cycler microscopy), respectively, and/or by means of a MELK robot system. For diseases, in which the characteristic CMP or the characteristic cluster of CMPs is already known and usually not patient-specific, it can be sufficient to determine the CMP or CMPs from a corresponding medical database. Alternatively or additionally, it can be provided that the CMP(s) characteristic of the disease is or are determined with the aid of a multi-epitope ligand cartography (MELK) and ICM method (imaging cycler microscopy), respectively, and/or by means of a MELK robot system. The MELK/ICM method and the MELK robot system, respectively, are for example known as such from the documents U.S. Pat. No. 6,150,173, DE 197 09 348 A1, EP 0 810 428 A1, DE 100 43 470 A1 and WO 02/21137 A2.

Alternatively or additionally, it is provided that the extracorporeal removal of the components by means of a MELK or ICM method and/or by means of a MELK robot system is monitored and/or controlled. Hereby, the desired depletion of the disease-specific components of the cell sample can be controlled and optionally be governed or regulated.

A particularly specific and thereby effective removal of the pathogenic and/or an excess of components from the cell sample with few side effects is possible in further configuration of the invention if the disease is a tumor disease and/or an inflammatory disease. Such diseases have a particularly high extent of cellular organization and thus can accordingly be specifically addressed within the scope of the present invention.

Further advantages arise in that the extracorporeal removal is performed by means of an apheresis method and/or by means of an apheresis device. Hereby, in particular pathogenic and/or an excess of components, which are at least predominantly in the blood and/or blood plasma of the diseased patient, can be particularly effectively removed from the blood and/or blood plasma with few side effects.

Therein, it has further proven advantageous if an unselective apheresis and/or a selective apheresis and/or a whole-blood apheresis and/or a photopheresis are performed as the apheresis method and/or that the apheresis device for performing at least one apheresis method is formed from the group of unselective apheresis, selective apheresis, whole-blood apheresis and photopheresis. This allows optimum adaptation to the respective disease and to the components characteristic of the concerned disease, respectively. In the extracorporeal apheresis, the blood of the patient can first be separated by means of a centrifuge in some implementations to enrich the pathogenic components with respect to non-pathogenic components. Similarly, it is possible to completely separate and/or substitute certain components. Within the scope of a photopheresis, the pathogenic components are exposed to controlled irradiation with light of certain wavelength(s) to achieve destruction or depletion. The wavelength(s), irradiation time and irradiation duration can be selected depending on the characteristics of the pathogenic components. For example, the optionally separated or fractionated cell sample can be exposed to a controlled UV-A and/or UV-B irradiation. Subsequent to the apheresis, the blood or blood plasma, in which the pathogenic components have been depleted or completely removed, can again be returned to the patient. Hereby, the treated cell sample is presented to the non-treated immune system and can effect an additional modulation of the immune system in some cases.

In a further advantageous configuration of the invention, it is provided that at least one streptamer and/or at least one antibody and/or at least one ligand, in particular a variable single-chain fragment (scFv) and/or a multivalent antibody fragment (scFv multimer), are used for extracorporeal removal. Hereby, a selection optimally adapted to the component to be removed can be performed. The at least one streptamer and/or the at least one antibody and/or the at least one ligand can basically be added to the cell sample and/or be used immobilized on a carrier material. Antibody fragments offer the advantage of a high binding affinity and avidity, respectively, and specificity for a wide spectrum of target structures and haptens. Single-chain fragments can additionally be cross-linked and expressed, respectively, as diabodies (60 kDa), triabodies (90 kDa), tetrabodies (120 kDa) etc., wherein different linker lengths between V domains are possible. In addition, a particular advantage is that molecules of 60-120 kDa increase the penetration of cells and have faster clearance rates than corresponding Igs (150 kDa). Furthermore, it can be provided that the antibody and/or ligand are a diabody, triabody, tetrabody, pentabody, hexabody, heptabody or octabody. In other words, it is provided that the antibody and/or ligand are formed mono-, bi-, tri-, quad-, pent-, hex-, hept-, oct-, enn- or multi-specific. This allows the cross-linking of two, three, four, five, six, seven or eight target structures or target proteins, wherein scFv multimers can be particularly precisely and individually adapted to the optionally patent-specific spatial arrangement of the target epitopes of certain combinatorial protein clusters (CMPs). The increased binding valence of scFv multimers results in a particularly high avidity.

Further advantages arise in that the at least one streptamer and/or the at least one antibody and/or the at least one ligand specifically bind and/or inactivate a leading protein characteristic of the disease. Hereby, a particularly efficient and extensive removal of the pathogenic and/or excess of components can be achieved and a correspondingly reliable treatment of the concerned disease can be allowed.

In a further advantageous configuration of the invention, it is provided that the disease is amyotrophic lateral sclerosis (ALS) and/or that the cell sample is a blood sample of the patient and/or that the at least one CMP includes one or more of the group of CD16, CD8, NeuN, Bax, Bcl2, CD11 b, CD138, CD16A, CD29, CD2, CD45RA, CD49d, CD54, CD56, CD57, CD58, CD62L, CD3, HLADR, immunoglobulin G, MHCII, MHCI, SIRT1, RAC1, BMX, GAK, JNK2, MAPKK6, OTUB2, PRKAR2A, SMAD2, SMAD4 and STAP2 and/or that the components are extracorporeally removed by means of at least one antibody and/or ligand, which bind at least one of the group of CD16, CD8, NeuN, Bax, Bcl2, CD11 b, CD138, CD16A, CD29, CD2, CD45RA, CD49d, CD54, CD56, CD57, CD58, CD62L, CD3, HLADR, immunoglobulin G, MHCII, MHCI, SIRT1, RAC1, BMX, GAK, JNK2, MAPKK6, OTUB2, PRKAR2A, SMAD2, SMAD4 and STAP2, and/or by means of a CD16 ectodomain shedding molecule. The amyotrophic lateral sclerosis (ALS) is based on an unstoppable and fast progressing irreversible paralysis of the musculature since certain neural cells controlling the musculature degenerate. Heretofore, over 50 clinical treatment studies have been performed, however, the therapy concepts of which have predominantly proven to be ineffective. With the aid of the method according to the invention, it is possible for the first time to extracorporeally remove or inhibit the pathogenic ALS-specific cells, whereby the disease progress can be stopped or at least considerably decelerated. The pathogenic components responsible for the ALS are aberrant T lymphocytes, which also express the CD16 receptor complex besides the CD8 receptor.

Therefore, they are characterized by CMPs, which contain CD8 and/or CD16 as leading proteins. The leading proteins can be colocalized or anti-colocalized with the further specified CDs and proteins. Accordingly, it has proven advantageous if the antibody and/or ligand used for removing the pathogenic components bind one, two, three, four, five or more of the group of CD16, CD8, STTP1, NeuN, Bax, Bcl2, CD11 b, CD138, CD16A, CD29, CD2, CD45RA, CD49d, CD54, CD56, CD57, CD58, CD62L, CD3, HLADR, immunoglobulin G, MHCII, MHCI, SIRT1, RAC1, BMX, GAK, JNK2, MAPKK6, OTUB2, PRKAR2A, SMAD2, SMAD4 and STAP2. In other words, it is provided that the antibody and/or ligand are formed mono-, bi-, tri-, quad-, pent-, hex-, hept-, oct-, enn- or multi-specific. The mentioned proteins form supra-individual and individual clusters in ALS-specific cells in different combinations and can therefore be extracorporeally therapeutically cross-linked and thereby blocked, whereby the ALS-specific cells lose their functionality. Hereby, a particularly specific treatment of ALS thereby with few side effects or even without side effects is allowed. Further advantages arise if the antibody and/or ligand bind at least CD16, CD8 and STTP1. Herein, STTP1 denotes the optionally patient-specific signal protein (signal transduction protein 1, e.g. kinase), which mediates the signal cascade extending from the cell surface to the cell nucleus, and couples on the surface of the cell together with the module “CD16a and CD8”, where it is co-localized with CD16a and CD8. STTP1 can be individually determined and monitored, respectively, for the concerned patient with the aid of the MELK and/or ICM technique (multi-epitope ligand cartography and imaging cycler microscopy, respectively) known per se. Furthermore, it can be provided that STTP1 is at least one protein from the group of RAC1, STAP2 and SMAD2. Further advantages arise if the antibody and/or ligand are recombinant, human or produced de novo.

Further advantages arise in that an antibody and/or ligand are used for extracorporeal removal, which binds two or more of the group of CD16, CD8, RAC1, STAP2 and SMAD2. Hereby, ALS-specific protein clusters can be blocked, which besides one or more cell surface proteins (e.g. CD8, CD16, CD45RA) are additionally directly associated with one or more signal chain molecules from the group of RAC1, STAP2 and/or SMAD2. RAC1 (Ras-related C3 botulinum toxin substrate 1) as a member of the RAC subfamily regulates a variety of cellular events, including the control of the cell growth, the cytoskeleton reorganization and the activation of protein kinases. STAP2 (signal-transducing adaptor protein 2) and SMAD2 (mothers against decapentaplegic homolog 2) are also part of the signal transduction chain and regulate the signal transduction and transcription of central signal paths in ALS-specific cells.

Alternatively, it is provided that the disease is prostate cancer and/or that the cell sample is prostate tissue and/or that the at least one CMP includes one or more of the group of CD26 and CD29 and/or that the components are extracorporeally removed by means of at least one antibody and/or ligand, which binds at least one of the group of CD26 and CD29. Hereby, it is possible for the first time to extracorporeally treat prostate cancer and to monitor the therapy. Therein, the invention is based on the realization that prostate cancer is characterized by CMPs, which contain CD26 and/or CD26 as leading proteins, which are optionally colocalized or anti-colocalized with further CDs and proteins, respectively.

Further advantage arise if the at least one CMP also includes CD44 and/or CD54 and/or CD138 and/or if the at least one CMP lacks in at least one of CD3, CD4, CD8, CD10, CD13, CD19, CD20, CD38, CD49d, CD58 and CD80. This allows a particularly reliable identification and monitoring of the removal of the pathogenic components of the cell sample responsible for prostate cancer. In particular, prostate cancer is present if the cell sample originates from the stroma and/or neoplastic epithelium of acini of prostate tissue and/or if at least 70%, preferably at least 75% of the CMPs contain CD26 and/or CD29 on the cell surface of at least one cell, and/or if at least 20%, preferably at least 35% of the CMPs include CD26 and/or CD29 on the cell surface of at least one cell and they lack in at least CD3, CD4, CD8, CD10, CD13, CD19, CD20, CD38, CD49d, CD58 and CD80.

Accordingly, the pathogenic components of the cell sample can be particularly reliably extracorporeally removed in further configuration of the invention that an antibody and/or ligand is used for the removal, which binds at least one or more of the group of CD26, CD29, CD44, CD54 and CD138.

Alternatively, it is provided that the disease is a cutaneous lymphoma, in particular mycosis fungoides, and/or that the cell sample is a skin sample and/or that the at least one CMP includes one or more of the group of HLA-DQ, CD2, CD3, CD4, CD7, CD8, CD10, CD13, CD18, CD18, CD26, CD29, CD36, CD44, CD45, CD49f, CD54, CD56, CD57, CD58, CD62L, CD71, CD80 and HLA-DR and/or that the components are extracorporeally removed by means of at least one antibody and/or ligand, which binds at least one of the group of HLA-DQ, CD2, CD3, CD4, CD7, CD8, CD10, CD13, CD18, CD18, CD26, CD29, CD36, CD44, CD45, CD49f, CD54, CD56, CD57, CD58, CD62L, CD71, CD80 and HLA-DR. Thereby, an extracorporeal treatment and therapy control of cutaneous lymphomas is allowed for the first time. Therein, the invention is based on the realization that cutaneous lymphomas are characterized by CMPs, which in particular contain HLA-DQ as a leading protein, which is colocalized or anti-colocalized with one or more of the mentioned CDs and proteins, respectively.

In a further advantageous configuration of the invention, the cell sample is again returned to the patient after partial or complete extracorporeal removal of the components, which have the at least one determined CMP.

A second aspect of the invention relates to a device, which is formed for performing a method according to the first inventive aspect. Therein, within the scope of the present invention, a device is to be understood by the expression “formed to”, which does not only have a basic suitability for performing such a method, but is specifically arranged and equipped to perform such a method. The features and advantages thereof resulting from it can be taken from the descriptions of the first inventive aspect, wherein advantageous configurations of the first inventive aspect are to be regarded as advantageous configurations of the second inventive aspect and vice versa.

Further advantages arise in that the device for performing at least one apheresis method is selected from the group of unselective apheresis, selective apheresis, whole-blood apheresis and photopheresis. In the unselective apheresis, which is also referred to as plasma exchange, the plasma is separated from the blood and completely substituted by means of the device. Mostly, humane blood products are used as replacement liquids. In the selective plasmapheresis (plasma perfusion), the cells carrying the protein cluster (CMP), which is characteristic of the disease of the patient, are separated from the plasma by filtration or adsorption by means of the device. The purified plasma can subsequently be returned to the patient. In the whole-blood apheresis (hemoperfusion), the cells carrying the protein cluster (CMP), which is characteristic of the disease of the patient, are for example affinity chromatographically directly filtered from the blood by means of the device. In the extracorporeal photopheresis, the cell sample or the blood of the patient can first be separated by means of a centrifuge of the device, leucocyte-enriched blood plasma can be collected by means of the device and exposed to controlled UV irradiation by means of an UV irradiation means of the device in an extracorporeal circulation. The wavelength of the UV irradiation is typically between 340 and 380 nanometers, for example 365 nanometers (UV-A). Typically, between 0.1 and 5 J/cm², for example 1.5 J/cm², can for example be provided as the average UV exposition of the leucocytes. The overall duration of the treatment is between 2 and 5 hours according to volume of the cell or blood sample. In the extracorporeal photopheresis, a light-activated pharmaceutical, for example methoxsalen, can be added to the sample by means of the device. Subsequent to the irradiation, the cell sample or the blood can be partially or completely returned to the patient. The advantage is in that the immune system of the patient is not impaired or only impaired to low extent such that an immune modulation of the non-irradiated immune system can be effected besides the direct cytostatic and cytotoxic effects on the irradiated cell or blood sample. The treatment frequency can be 4 to 20 cycles in intervals of respectively 2-20 days. The intervals between individual treatments can increase over time. The treatment success can be effected via determination of the concentration decrease of the cells, which carry the protein cluster (CMP), which is characteristic of the disease of the patient. A further advantage of apheresis methods is in the comparatively low costs.

According to an Australian analysis, the costs per ALS patient and year currently amount to about 750,000 to 1 million of Euros. Since one is able to at least stop ALS in the early stage for example with a photopheresis treatment, the development of progressive severe disabilities and the stresses associated therewith can be prevented with up to about 26 treatments per year, which can be realized for a fraction of the previous annual costs, which also results in considerable savings in the health system besides the unquantifiable gain of lifetime and quality for the patient.

A third aspect of the invention relates to a streptamer and/or an antibody and/or a ligand for use in a method according to the first inventive aspect. The features and advantages thereof resulting from it can be taken from the descriptions of the first inventive aspect, wherein advantageous configurations of the first inventive aspect are to be regarded as advantageous configurations of the third inventive aspect and vice versa. The streptamer, the antibody and/or the ligand can be coupled to an adsorber and/or a chromophore in further configuration. Hereby, they are in particular suitable for the use within the scope of a photopheresis with wavelengths matched to the absorber and the chromophore, respectively. Alternatively or additionally, the streptamer, the antibody and/or the ligand can be coupled to a marker, in particular a magnetic marker (magnetic cell separation). This allows simple extracorporeal separation of pathogenic components bound to the streptamer/antibody/ligand by affinity chromatography, for example with the aid of so-called microbeads. They are about 50 nanometers large magnetic particles, which are bound to the streptamer, the antibody and the ligand, respectively. Streptamer/antibody/ligand recognizes disease-specific CMP(s) on the surface of pathogenic components (cells) and thereby ensures binding of the microbeads to this cell population. Upon passage of the cell sample through a column, which is surrounded by a strong magnetic field, the cells marked with the microbeads are retained. In this manner, one obtains only unmarked and thereby non-pathogenic cells in flushing the column such that the marked cell population and thereby the components characteristic of the disease have been removed from the original cell mixture. After removing the magnetic field, the marked cell population can additionally also be obtained by flushing the column and is thereby available for further experiments. By repeated treatment of a cell mixture with various microbeads, a so-called fractionated separation, thus, the separation into multiple cell populations, is also possible. The streptamer, the antibody and/or the ligand can be coupled to a fluorescent dye, a quantum dot, a radioactive marker, a spin marker and/or a tag for a further antibody/ligand and/or an enzyme in further configuration.

Further features of the invention are apparent from the claims, the figures and the description of figures. The features and feature combinations mentioned above in the description as well as the features and feature combinations mentioned below in the description of figures and/or shown in the figures alone are usable not only in the respectively specified combination, but also in other combinations without departing from the scope of the invention. Thus, implementations are also to be considered as encompassed and disclosed by the invention, which are not explicitly shown in the figures and explained, but arise from and can be generated by separated feature combinations from the explained implementations. Implementations and feature combinations are also to be considered as disclosed, which thus do not comprise all of the features of an originally formulated independent claim. There shows:

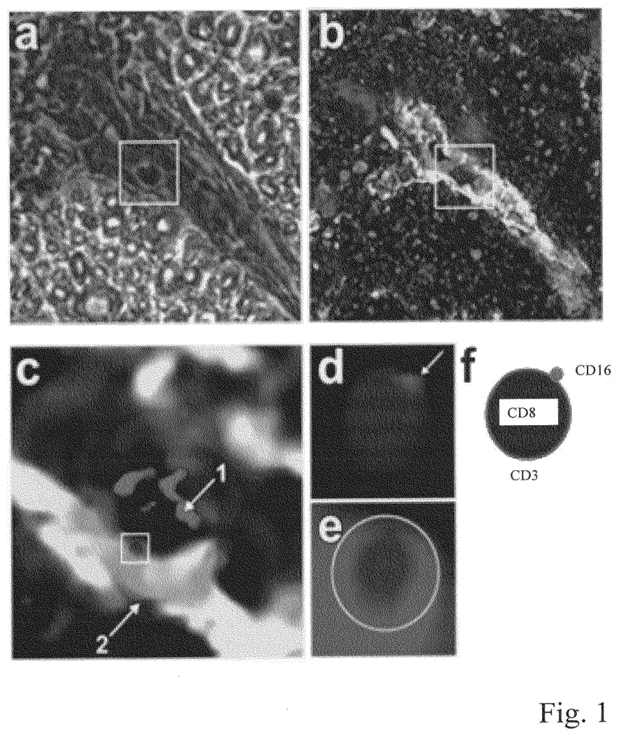

FIG. 1a to 1f various toponome images of ALS-specific, abnormal cells;

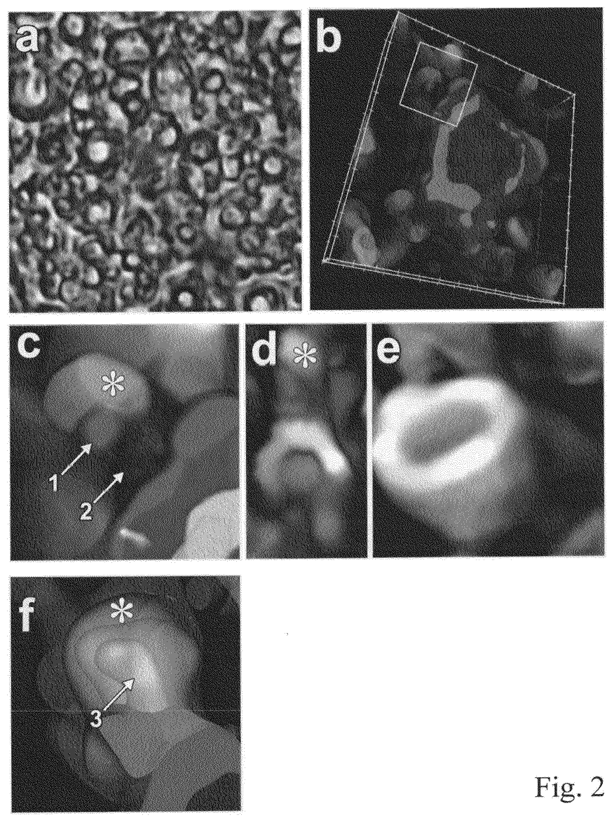

FIGS. 2a to 2f the stage process of these abnormal cells, which results in the ALS-specific syndrome;

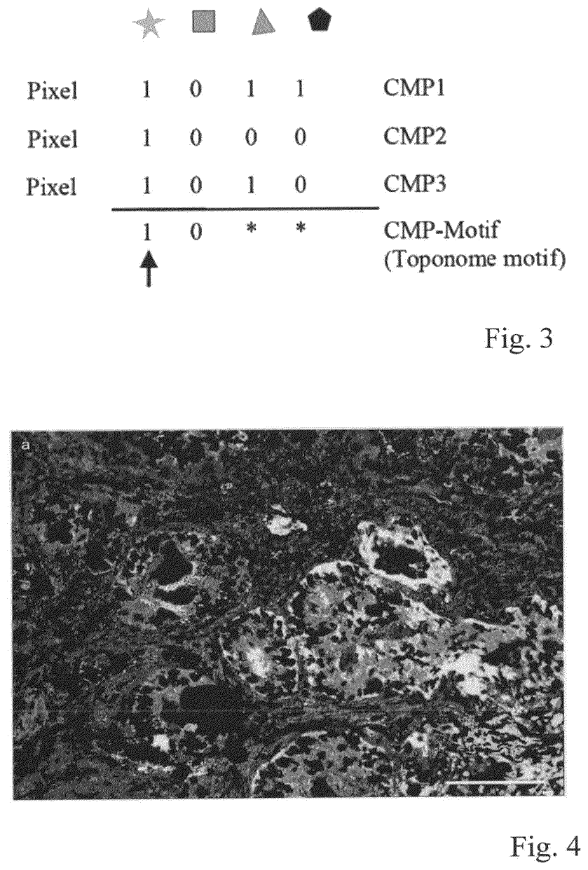

FIG. 3 a schematic representation of the topologic hierarchy of proteins within a toponome;

FIG. 4 a toponome map, which shows the position of the 30 most frequent from more than 2,000 different CMPs in prostate cancer;

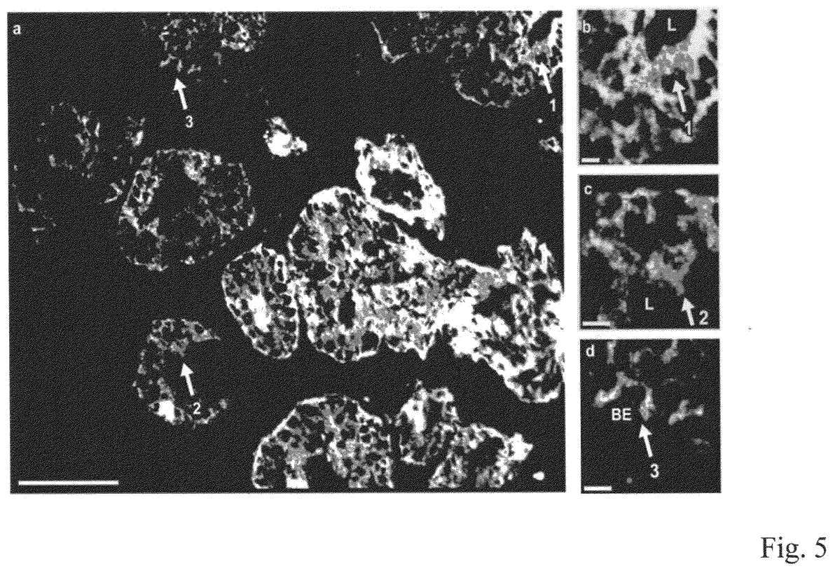

FIG. 5 a representation of the selective expression of the prostate cancer-specific CMPs at subcellular locations of neoplastic prostate acini;

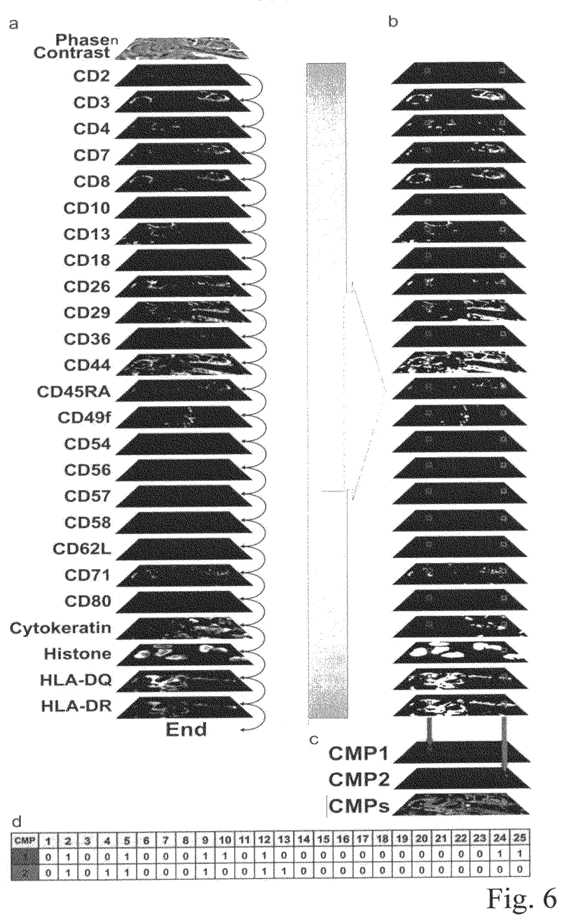

FIG. 6 an illustration of the cyclic localization of 25 biomolecules and the following determination of the CMPs with the aid of the MELK/ICM method;

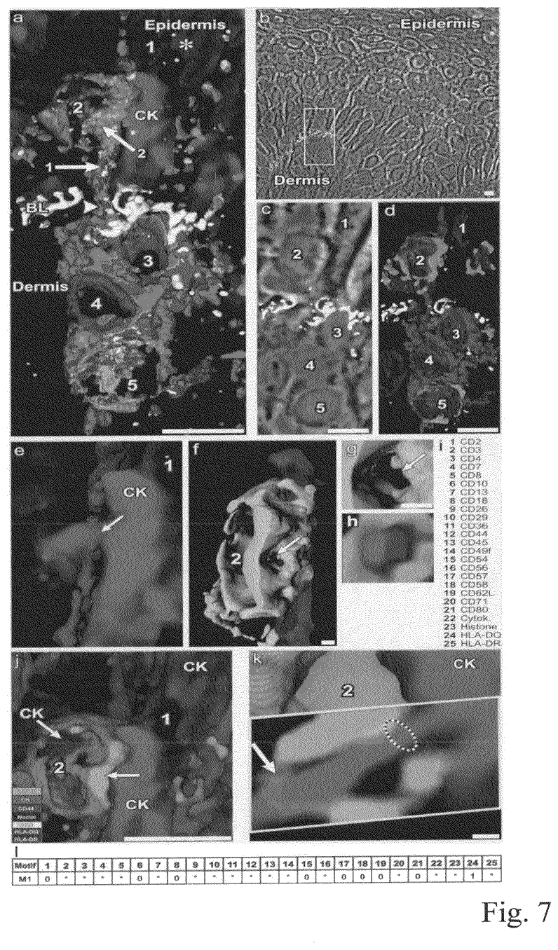

FIG. 7 toponome depictions to the tissue organization of a cutaneous lymphoma;

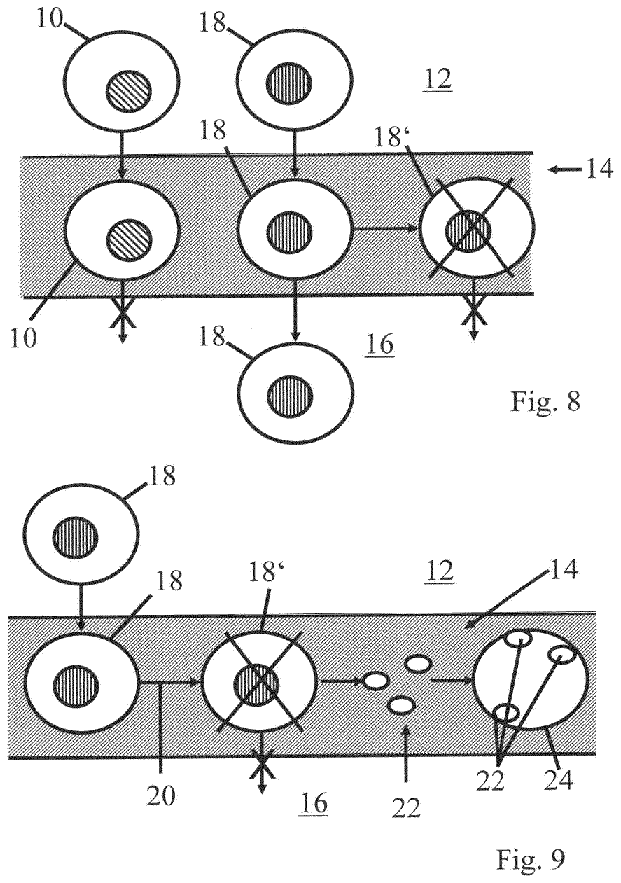

FIG. 8 a schematic representation of the pathomechanism of the ALS; and

FIG. 9 a schematic representation of a therapy of ALS.

### MECHANISMS OF THE ALS IN THE TOPONOME

Synopsis of the ALS toponome: Analyses of the cell surfaces of immune cells of the blood have shown that a specific abnormal toponome occurs in ALS. An essential feature is the existence of aberrant T lymphocytes, which also express the CD16 receptor complex besides the CD8 receptor. Exactly the same cell forms are found in the morphologically well preserved ALS postmortal tissue within post-capillary venules of the spinal pyramidal tract (FIG. 1a, b: box). Detailed 3D toponome resolution shows that these cells express the CD16 complex (FIG. 1, arrow 1), are polarized and show a “leading edge” with CD3 positive membrane, which penetrates through the endothelial cell layer of the blood vessel (FIG. 1c; arrow 2), while CD3 positive vesicles are intracellularly transported to the “leading edge” (FIG. 1c; box). Individual vesicles contain complexes of CD8/CD16 (FIG. 1d, e, f). These CD8/CD16 complexes are required for the transmigration of these cells through the endothelial cell layer via “vesicle budding” in the cell surface by “forward transport” to advance the transmigration as part of an abnormal “homing code” (reference code). CD16 is also referred to as Fc region receptor III-A and FcγIII, respectively. After the transmigration, this complex is degraded since CD8 positive T cells in the parenchyma of the pyramidal tract do not have the CD16 complex. Instead, they are found as individual cells between the myelinated neurons (FIG. 2a, CD8+CD16—blue cell). Greater 3D magnification of this cell exhibits a cell extension, which has penetrated deeply into the surface of a morphologically intact axon (FIG. 2b, box; FIG. 2c: magnification of the box: asterisk=morphologically intact axon body). This cell extension expresses CD3 and CD8 (FIG. 2; arrows 1, 2). A geometrically exact 3D calculation results in the cell extension in FIG. 2f (arrow 3) and the axon in FIG. 2f (marked with asterisk). A similar cell extension is found based on the same cell in FIG. 2d (lower image half): Here, the cell extension (red) obviously has penetrated the intact myelin sheath (white). For comparison hereto, FIG. 2e shows an intact myelin sheath.

To sum up: CD16 is required to control the abnormal “homing” of aberrant CD8 cells into the pyramidal tract. When this homing process is completed, the cells degrade (lose) the CD16 complex, then migrate between the myelinated neurons as CD8+CD16 cells, penetrate through the myelin sheaths and axotomize neurons. This process is obviously primarily autonomous since the cells are never observed in context with cell “debris”, thus signs of primary neuronal degradation. Therefore, the cells play a primary pathogenetic role. This interpretation is supported by the fact that the down-regulation of CD16 results in a stop of the disease progression: if CD16 is no longer available as in FIG. 1f, the endothelial homing operation can no longer be carried out. A part of the (sporadic) ALS can therefore be described as a disease of the immune cell toponome, which generated the ALS-specific pyramidal tract lesions (axotomy) by means of a highly selective “homing code”. Many genetic findings and protein aggregations, which were described in ALS, also have been described in experimental axotomy, such that the axotomy mediated by immune cell toponome explains these findings.

Coexpression Pattern

The invasive cells according to FIG. 1c further coexpress the following molecules:

### Bax, Bcl2, CD11 b, CD138, CD16A, CD29, CD2, CD45RA, CD49d, CD54, CD56, CD57, CD58, CD62L, CD8, HLADR, Immunoglobulin G, MHCII, MHCI, NeuN, SIRT1, RAC1, BMX, GAK, JNK2, MAPKK6, OTUB2, PRKAR2A, SMAD2, SMAD4 and STAP2.

NeuN (Fox-3, Rbfox3 or hexaribonucleotide binding protein-3) is a neuronal, nuclear antigen, which is normally used as a biomarker for neurons.

The nuclear coexpression of NeuN and CD49d as well as the cytoplasmic expression of immunoglobulin G (IgG) together with the CD16 carrying vesicles from FIG. 1d-f) and the cell surface expression of CD8 and CD3 show that this cell has an abnormal differentiating status: A cell with features of a T cell (CD3, CD8), a neuronal cell (NeuN in the cell nucleus), a monocyte (CD16) and a B lymphocyte (IgG).

Proteins, which are partially patient-specific, that is colocalized with CD8 and/or CD16 in ALS-indicative cells with individually different probability or frequency, are indicated in table 1. Thus, all of these proteins represent therapeutic target structures (targets) individually or in combination with CD8 and/or CD16, since deactivation of these proteins results in collapse of the intracellular information flow and thereby in functional loss of the aberrant cells.

TABLE 1

| | Official | |
|---|---|---|
| Protein | symbol | Official name |
| BMX | [BMX] | BMX non-receptor tyrosine kinase |
| GAK | [GAK] | cyclin G associated kinase |
| JNK2 | [MAPK9] | mitogen-activated protein kinase 9 |
| MAPKK6 | [MAP2K6] | mitogen-activated protein kinase |
| | | kinase 6 |
| OTUB2 | [OTUB2] | OUT deubiquitinase, ubiquitin aldehyde |
| | | binding 2 |
| PRKAR2A | [PRKAR2A] | protein kinase cAMP-dependent type II |
| | | regulatory sub-unit alpha |
| RAC1 | [RAC1] | ras-related C3 botulinum toxin |
| | | substrate 1 (rho family, small GTP |
| | | binding protein Rac1) |
| SMAD2 | [SMAD2] | SMAD family member 2 |
| SMAD4 | [SMAD4] | SMAD family member 4 |
| STAP2 | [STAP2] | signal transducing adaptor family member 2 |
| SIRT1 | [SIRT1] | nicotinamide adenine dinucleotide-dependent |
| | | protein deacetylase |

The cell from FIG. 2c,f), which expresses a CD16 negative phenotype after invasion of the cell from FIG. 1 as substantiated above, remains positive for NeuN.

Significance: The above described invasive cell is detectable as an ALS-specific cell with the above mentioned features in blood or in tissue samples by spatial toponome analysis. Since the invasion behavior thereof into the pyramidal tract is known, as set forth above, and since it is further known that the cell obviously transforms to a NeuN positive T cell by phenotype switching (CD16-), which axotomizes neurons, the detection of cells of the CD16/CD8 phenotype from FIG. 1c is indicative of the process of axotomy. From this, it follows that the cell invasion of the cell from FIG. 1c has to be therapeutically prevented by molecular blocking or lysis to prevent the axotomy and to stop the progress of ALS.

The described two-step process (homing of aberrant “NeuN” positive blood cells into the pyramidal tract, the transformation thereof into “NeuN” positive T cells, which injure the surfaces of neural cells) presents a violation of basic rules of the cooperation of cells of various germ layers: In intact systems, these rules are based on the fact that cells from different germ layers comply with the rule of the non-injury of the mutual cell surfaces. In case of ALS, the transformed T cell-like cells (germ layer mesenchyme) injure the surfaces of neurons (germ layer ectoderm). Thereby, a physical trans-cellular cross-linking of the two germ layer functions with the consequence of the interruption of the neural pathways to the arbitrary musculature occurs. The fact of the expression of “NeuN” in the cell nucleus of the acting cells (immune cells in the circulation and after invasion into the neural system of the pyramidal tract) means that these cells follow a program of aberrant neuronal stem cells, since NeuN is only expressed in neurons under physiological conditions. Thereby, NeuN can be used as a marker for the presence of ALS causing cells, that is for diagnosis of the ALS.

In order to block the molecular “program” of these aberrant cells, that is as a therapy measure for treating a patient suffering from ALS, various extracorporeal methods lend themselves to extracorporeally remove the pathogenic cells expressing the leading protein CD16.

Hereto, the blood of the patient can be subjected to therapeutic apheresis, in which the pathogenic cells are removed from the blood with the aid of preferably immobilized antibodies and/or ligands, which bind CD16. Alternatively or additionally, immobilized antibodies and/or ligands, which bind CD8 and/or RAC1 and/or STAP2 and/or SAMD2, can preferably also be used. Similarly, it is possible to use multiple different antibodies and/or ligands, which are arranged together and/or one after the other viewed in flow direction of the blood, such that a multi-stage depletion of the pathogenic components of the blood results. Alternatively or additionally, it is possible to fractionate the blood before extracorporeal removal of the pathogenic cells, for example by centrifugation.

Alternatively or additionally, it is possible to treat the blood by photopheresis, wherein the optionally fractionated blood is irradiated with light of certain wavelength(s) for a predetermined time to inactivate the pathogenic cells.

A therapeutic depletion of “ALS cells”, that is of cells, which carry the ALS-specific CMP, in the blood of patients, which were affected with ALS, was achieved as follows:

First, blood samples of multiple patients, which were suspected of being affected with amyotrophic lateral sclerosis (ALS), were examined with the aid of the imaging cycler technology (MELK/ICM method) with a combinatorial molecular resolution of up to 4.5×10⁴⁸¹. Hereto, a toponome imaging system (TIS) of the company ToposNomos Ltd. (Munich, Germany) was used. Patients, in which so-called “ALS cells”, that is cells with ALS-typical CMPs, were detected, were categorized as affected with ALS, wherein the individual patients each had different progression durations of the disease. The identified patients were then subjected to a photopheresis treatment. Photopheresis allows performing a specific depletion method such that the number and concentration of “ALS cells” in the blood of the patients can be considerably, that is down below the detection limit, reduced. As it has turned out, the patients subjectively felt better if more than 1 liter of blood volume, that is at least 2 and preferably up to 5 liters of blood volume, were passed through the device for treatment within the scope of a photopheresis cycle.

As it could be assessed by the initial examination and subsequent progression controls with the aid of the imaging cycler technology, the cell number of the “ALS cells” in the blood very exactly correlates with the progression speed of the disease. For example, in a patient with about 50 millions of “ALS cells” per liter of blood, the disease proceeded less than half as fast as in a patient with about 140 millions of “ALS cells” per liter of blood. This as well as the above discussed realization that exactly these “ALS cells” migrate into the pyramidal tract (pathologic homing) after conversion of the cell surface, where they axotomize the axons in the pyramidal tract, represent a further evidence that the “ALS cells” detected in the blood are the crucial pathogenetically relevant cells and thereby a central target of the ALS treatment.

The therapeutic depletion of these “ALS cells” in the blood always succeeded very securely by means of photopheresis in all of the patients treated up to now. Already after few photopheresis cycles, typically about 2 to 6 cycles, virtually no “ALS cells” could be detected in the blood anymore. One observes particular effects in initial stages of the ALS disease. Thus, the disease is no longer progredient in the treated patients since about 5 months or longer according to previous observations, the electrophysiological parameters (EMG etc.) did not show any further progression and the treated patients felt considerably better. In particular, no or hardly any florid EMG activities can be assessed in the treated patients.

As it appeared in some patients, the “ALS cells” can return in the blood in pauses between the photopheresis cycles. The returning “ALS cells” can again be completely brought to depletion by means of photopheresis. According to previous knowledge, this procedure can be repeated any number of times such that at least progress of the ALS disease can be stopped or considerably decelerated. In addition, it was observed that the pathogenic ALS-specific combinatorial CMP motifs of the “ALS cells” decreased at least by a factor of 100 after the return thereof in the blood. Similarly, the number of the disease-specific molecular combinatorics on the cell surface (the so-called combinatorial molecular phenotypes, CMPs, with code (=1) and anti-code (=0) (Schubert W. et al., Nat. Biotechnol 2006)) of the individual “ALS cells” considerably decreased. This effect stably appears over the period of time examined up to now. Since cell surface CMPs on the surface of these pathologic “ALS cells” “decelerate” the rolling phenomenon until stop in the postcapillary, the site of invasion, the decrease of the number of these CMPs induced by photopheresis will deteriorate the pathologic homing after rolling and thereby stop or at least greatly decelerate the disease process at the pathogenesis site of the invasion.

Further possibilities to extracorporeally remove the pathogenic cells with the aid of apheresis methods include:

1. Use of molecules (antibodies/ligands), which bind and/or block/inhibit IgG (1/3) and block/inhibit the physiological mechanism of the IgG mediated activation of CD16, respectively. An example hereto are antibodies against IgG and (optionally recombinant) streptococcus protein G;

2. Blocking (e.g. by cross-linking) of the ALS-specific cluster CD16a+CD8+STTP1, for example by means of at least one bi-, tri- or multi-specific antibody (recombinant, human, de novo etc.), which is preferably present bound to a matrix. Herein, STTP1 denotes the optionally patient-specific signal protein (signal transduction protein 1, e.g. kinase), which mediates the signal cascade originating from the cell nucleus and is coupled on the surface of the cell together with the module “CD16a and CD8” and is colocalized with CD16a and CD8, respectively. STTP1 can be individually determined and controlled, respectively, for the concerned patient with the aid of the MELK and/or ICM technique (multi-epitope ligand cartography and imaging cycler microscopy, respectively) known per se.

STTP1 can for example be one or more proteins from the group of RAC1, STAP2 and/or SMAD2 and/or another protein from the signal transduction chain of ALS cells. A corresponding antibody, for example a tri-specific anti-CD16a-CD8-STTP1 antibody, can then also be produced via production methods known per se and be used for therapy of the concerned patient. Since such a tri-specific antibody has an extremely high selectivity for abnormal, ALS-specific cells, such a treatment is particularly low in side effects;

3. Use of molecules, which effect the proteolytic separation of the extracellular CD16 domains (ectodomains) (so-called shedding and ectodomain shedding, respectively). An example hereto is the use of preferably immobilized Adam17 (metallopeptidase domain 17), which is also called TACE (tumor necrosis factor α converting enzyme). Adam17 is a 70 kDa enzyme, which belongs to the ADAM protein family of disintegrins and metalloproteases. Adam17 and the respective CD16 molecule capable of shedding can optionally be coupled to a mono-, bi-, tri-or multi-specific antibody (see item 2) to advantageously increase its specifity;

4. Use of at least one mono-, bi-, tri- or multi-specific anti-CD16 antibody (recombinant, human, de novo etc), which is preferably immobilized on a matrix material;

5. Use of at least one mono-, bi-, tri- or multi-specific anti-NeuN antibody (recombinant, human, de novo etc.), which is preferably immobilized on a matrix material;

6. Use of streptamers and/or magnetic beads for removing the pathogenic CD16 cells.

Therein, it can be provided that the pathogenic cells are not or not completely removed from the blood sample, but are extracorporeally inactivated such that they lose their biological functionality.

The above mentioned compounds (drugs) can basically be administered individually or in any combination within the scope of an apheresis method. The efficacy and the extent of removal should be monitored and optionally adapted by regular control. The control can for example be performed with the aid of the above described diagnostic method. Upon successful removal, significantly less or preferably no abnormal ALS-specific cells (see above) anymore can usually be assessed in the cell or blood sample of the concerned patient.

Thereby, numerous ALS-specific supra-individual and/or individual therapeutic options generally arise. A supra-individual therapy can be directed to the ALS-specific surface proteins (CDs) and surface protein clusters, respectively, which are cross-linked, inhibited or bound to a matrix. In addition, central signal chain molecules of ALS cells can optionally be cross-linked, inhibited or bound to a matrix. For example, the cell surface proteins CD8/CD16A/CD45RA can optionally be extracorporeally blocked together with the signal chain molecule RAC1, since they occur directly associated exclusively in ALS cells. Such a therapy can for example be effected with the aid of bi-, tri- and/or tetra-specific antibodies and by corresponding single-chain variable fragments (scFv) or multivalent antibody fragments (scFv multimers), respectively, that is by so-called diabodies, triabodies or tetrabodies.

Individual targets, which can be therapeutically addressed alternatively or additionally to the above indicated ALS-specific clusters, include individual clusters of CD16/CD8 with the molecules STAP2 and/or SMAD2 as well as optionally combinations of the molecules indicated in table 1 together with the CD8/CD16 cluster or also as isolated clusters without CD8/CD16. These clusters too can be deactivated with the aid of optionally multi-specific antibodies and/or antibody fragments.

FIG. 3 shows a schematic representation of the topologic hierarchy of proteins within a toponome. The proteins are symbolized with the symbols asterisk, square, triangle and pentagon. One recognizes that the protein symbolized with asterisk occurs in all three CMPs 1-3, such that this protein is a leading protein (L, 1, true).

In contrast, the protein identified by square occurs in none of the CMPs 1-3 such that this protein is an anti-colocalized protein (A—absent, 0, false). The other proteins (triangle, pentagon) are variably associated with the leading protein and thus represent so-called wildcard proteins (W, *).

FIG. 4 shows a toponome map, which shows the position of the 30 most frequent from more than 2,000 different CMPs in prostate cancer. The position of these CMPs in the tissue is shown as an overlay with a CD138 fluorescent signal. FIG. 4 illustrates the position of these CMPs by their different colors (respectively grey scales), which are oriented at the CD138 fluorescent signal. Relevant CMPs are shown in the following table 2 and have CD26 and CD29 as the leading proteins (=L), while CD3/CD4/CD8/CD19/CD20/CD44/CD80 are absent in all of these CMPs (anti-colocalized, A, 0), and CD10/CD13/CD38/CD49d/CD54/CD58/CD138 are variably associated (W, *). As mentioned, this results in the symbol code (L, A, W) for the protein clusters (CMP motif). Thereby, CD26 and CD29 could be identified as the leading proteins on the cell surfaces of cells of prostate tissue of a patient, who is affected with prostate cancer.

FIG. 5 shows a representation of the selective expression of the prostate cancer-specific CMPs at subcellular locations of neoplastic prostate acini. FIG. 5 illustrates the selective expression of CMP1 at subcellular locations of neoplastic prostate acini. CMP1 is the most frequent CMP within the CMP motif of the table 2. FIG. 5a shows an overview, in which arrow 1 and arrow 2 identify apical locations of secretory cells, while arrow 3 identifies projections at the basolateral location of an acinus. FIG. 5b-c show details, which are identified by arrows. One recognizes the annular arrangement of the CMP1 (brown color) with the cell surface fluorescent signal of CD133 (white). “BE” means basal epithelial cell layer (bars: 5 a: 100 μm; 5 b-d: 10 μm).

Accordingly, prostate cancer can be treated analogously to the ALS by extracorporeal removal of components and cells, which have CMPs, which include at least CD26 and/or CD29 as disease-specific leading proteins. Hereto, an apheresis and photopheresis method, respectively, can for example be used, in which the CD26/CD29 expressing cells are separated and/or inactivated or destructed in the above described manner.

FIG. 6 shows an illustration of the cyclic localization of 25 biomolecules and the following determination of the CMPs with the aid of the MELK/ICM method. FIG. 6a shows an optical plane of 20 commonly mapped planes in the z-direction and serves for illustrating the fluorescent signals of the biomolecules specified in FIG. 7. FIG. 6b illustrates the binarization of the parallel shown primary signals of FIG. 6a. FIG. 6c shows 2D maps of the resulting combinatorial molecular phenotypes (CMPs), which are calculated for all of the data points from the binary dataset (FIG. 6b). FIG. 6d shows two exemplary CMPs from the data points (FIG. 6b).

FIG. 7 shows toponome depictions to the tissue organization of a cutaneous lymphoma (bars: in a-d, j: 10 μm, in f, g, k: 1 μm). FIG. 7a shows a 3D co-mapping of 3213 CMPs (from 7161 CMPs) within an area of a cryosection of a tissue sample of a patient affected with a cutaneous lymphoma (mycosis fungoides) according to FIG. 7b (boxed area). Different CMPs are coded with different colors. FIG. 7b shows a phase contrast image of the cryogenic skin section, in which nuclei for histones are blue colored, while the basal lamina for CD49f is white colored. FIG. 7c shows an enlargement of the boxed area (FIG. 7b) and is identically oriented as FIG. 7d, in which the leading proteins of the CMPs of FIG. 7a are shown. One recognizes an elongated multi-cellular complex of five cell types (cells 1-5 in a, d, c). An elongated cell projection (arrow 1 in a) of the cells 3, 4, 5 extends from the dermis via the basal lamina (a, BL) into the epidermis, where it is close to CD8+/CD3+T cells (cell 2 in a, c, d, brown color in d and adjacent keratinocyte (cell 1 in a, c, d)). FIG. 7e shows details of a suprabasal part of this structure from different viewing angles (e, f, g, h). A cytokeratin containing cell projection (CK) extends away from the keratinocyte (e, arrow) and projects through the CD8+/CD3+T cell surface (compare h, which shows CK in green; compare g, which shows the same without CK). FIG. 7k shows a transverse virtual anatomic section through the CD8+/CD3+T cell (cell 2 in j). The projection of the keratinocyte penetrates into the T cell (arrow). FIG. 7i shows a list of the co-mapped molecules. FIG. 7l specifies the CMP motif characteristic of the cutaneous lymphoma.

### L: HLA-DQ

### A: CD2, CD10, CD18, CD54, CD57, CD58, CD62L, CD80

### W: CD3, CD4, CD7, CD8, CD13, CD26, CD29, CD36, CD44, CD45, CD49f,

CD56, CD71, HLA-DR, cytok., histones.

Accordingly, the cutaneous lymphoma can be treated analogously to the ALS by extracorporeal removal of components and cells, which have CMPs, which at least include HLA-DQ as the disease-specific leading protein. Hereto, an apheresis and photopheresis method, respectively, can for example be used, in which the HLA-DQ expressing cells are separated and/or inactivated or destructed in the above described manner.

Therefore, the method can basically include a combination of the toponome technology with methods of isolating cells from body fluids and/or tissues. By application of the toponome technology, cells can be extremely accurately classified by co-mapping thousands of multi-protein complexes. In this manner, the disease-specific cells and CMPs, respectively, are found. The concerned cells can then be specifically isolated with methods known per se and be further used for diverse purposes, e.g. cloning, extracorporeal expansion, retransfer into tissues and blood, respectively, therapeutic applications etc. A particular aspect is the isolation of disease-specific cells from the blood circulation, which migrate into organs as autoimmune cells or aberrant cells of the immune system, where they specifically destruct tissue structures. In the latter case, e.g. the isolation of such cells from the blood circulation by a therapeutic apheresis/photopheresis would be a much targeted measure with the aim of stopping the disease progression since the disease-specific cells can be exactly predefined by high-dimensional toponome mapping such that they can be specifically removed via isolation from the circulation based on antibodies. Via consecutive monitoring by means of toponome mapping of the blood, the efficiency of the method can be controlled. A further advantage is in the use of such cells for the development of medicaments for selective blocking/elimination of such cells or in case of stem cells for reinfusion into the organism (e.g. therapeutic organ regeneration or tumor therapy) or for use for the development of diagnostics or for the therapy of the amyotrophic lateral sclerosis (ALS) or other diseases by removal of pathogenic cells from the blood circulation to prevent the biological mechanism thereof, in case of ALS the invasion into the motor neuron system and the neurotoxic damaging mechanisms thereof.

FIG. 8 shows a schematic representation of the pathomechanism of the ALS. A normal or healthy T cell 10 first matures in an extrathymic environment 12 (skin) and is released into the blood stream 14. Due to lacking homing codes, the normal T cell 10 cannot (and is not to) penetrate into the pyramidal system 16, which is responsible for the minute motor activity and the arbitrary motor activity in mammals. In contrast, “ALS cells” 18 represent aberrant T cells with a defective homing code, which can be characterized by the above described CMPs. Accordingly, ALS cells 18 circulating in the blood can penetrate into the pyramidal system 16 from the blood, where they axotomize neurons, which results in a degeneration of the motoric neural system and thereby in the ALS-typical disease pattern with progressive paralysis of the patient. Therefore, the aim of an ALS treatment has to be preventing the development of ALS cells 18 and/or the invasion of already present ALS cells 18 into the pyramidal system 16. For example, ALS cells 18 can be deactivated by blocking the leading proteins and/or by induction of apoptosis (deactivated ALS cells 18′), whereby they lose their biological functionality and can no longer migrate into the pyramidal system 16.

FIG. 9 shows a schematic representation of a therapy of ALS. As already mentioned, aberrant ALS cells 18 mature in an extrathymic environment 12 (skin) and are released into the blood stream 14. By a therapeutic treatment 20 of the blood 14, for example by photopheresis and/or by deactivation of the ALS leading proteins CD8 and CD16 by cross-linking with bi- or multi-specific antibodies, the ALS cells 18 circulating in the blood stream 14 become apoptotic (deactivated ALS cell 18′) and can no longer migrate into the pyramidal system 16. Apoptotic bodies 22 of the deactivated ALS cells 18′ evolve. These apoptotic bodies 22 are received and degraded by adjacent cells 24 (phagocytosis). Therein, the apoptotic bodies 22 are also presented to the immune system. According to current state of knowledge, it is to be assumed with high probability that an endogenous immune response against the aberrant ALS cells 18 can also be induced by this mechanism such that the extrathymic maturation of the aberrant ALS cells 18 can at least partially be inhibited and the number of the recreated ALS cells 18 decreases.

The parameter values indicated in the documents for the definition of process and measurement conditions for the characterization of specific characteristics of the inventive subject matter are to be considered as encompassed by the scope of the invention also within the scope of deviations—for example due to measurement errors, system errors, weighing errors, DIN tolerances and the like.

1. A method for extracorporeal removal of pathogenic and/or an excess of components from a cell sample of a human or animal patient suffering from a disease including the following steps:
a) determining at least one protein cluster (CMP), which is characteristic of the disease of the patient;
b) providing the cell sample of the patient; and
c) extracorporeal removal of components having the at least one determined CMP from the cell sample.

2. The method according to claim 1, wherein:
a body fluid, and/or a tissue sample of the patient are provided as the cell sample.

3. The method according to claim 1, wherein the at least one CMP is determined based on a database and/or by means of a multi-epitope ligand cartography (MELK) and ICM method (Multi-epitope ligand cartography/imaging cycler microscopy), respectively, and/or by means of a MELK robot system and/or that the extracorporeal removal of the components is monitored and/or controlled by means of a MELK and ICM method, respectively, and/or by means of a MELK robot system.

4. The method according claim 1, wherein the disease is a tumor disease and/or an inflammatory disease.

5. The method according to claim 1, wherein the extracorporeal removal is performed by means of an apheresis method and/or by means of an apheresis device.

6. The method according to claim 5, wherein an unselective apheresis and/or a selective apheresis and/or a whole-blood apheresis and/or a photopheresis are performed as the apheresis method and/or that the apheresis device for performing at least one apheresis method is formed from the group of unselective apheresis, selective apheresis, whole-blood apheresis and photopheresis.

7. The method according to claim 1, wherein at least one streptamer and/or at least one antibody and/or at least one ligand, in particular a variable single-chain fragment (scFv) and/or a multivalent antibody fragment (scFv multimer), are used for extracorporeal removal.

8. The method according to claim 7, wherein the at least one streptamer and/or the at least one antibody and/or the at least one ligand specifically bind and/or inactivate a leading protein characteristic of the disease.

9. The method according to claim 1, wherein the disease is amyotrophic lateral sclerosis (ALS) and/or that the cell sample is a blood sample of the patient and/or that the at least one CMP includes one or more of the group of CD16, CD8, NeuN, Bax, Bcl2, CD11b, CD138, CD16A, CD29, CD2, CD45RA, CD49d, CD54, CD56, CD57, CD58, CD62L, CD3, HLADR, immunoglobulin G, MHCII, MHCI, SIRT1, RAC1, BMX, GAK, JNK2, MAPKK6, OTUB2, PRKAR2A, SMAD2, SMAD4 and STAP2 and/or that the components are extracorporeally removed by means of at least one antibody and/or ligand, which bind at least one from the group of CD16, CD8, NeuN, Bax, Bcl2, CD11b, CD138, CD16A, CD29, CD2, CD45RA, CD49d, CD54, CD56, CD57, CD58, CD62L, CD3, HLADR, immunoglobulin G, MHCII, MHCI, SIRT1, RAC1, BMX, GAK, JNK2, MAPKK6, OTUB2, PRKAR2A, SMAD2, SMAD4 and STAP2, and/or by means of a CD16 ectodomain shedding molecule.

10. The method according to claim 9, wherein an antibody and/or ligand is used for extracorporeal removal, which binds two or more from the group of CD16, CD8, RAC1, STAP2 and SMAD2.

11. The method according to any one of claim 1, wherein the disease is prostate cancer and/or that the cell sample is prostate tissue and/or that the at least one CMP includes one or more of the group of CD26 and CD29 and/or that the components are extracorporeally removed by means of at least one antibody and/or ligand, which bind at least one of the group of CD26 and CD29.

12. The method according to claim 11, characterized in that the at least one CMP also includes CD44 and/or CD54 and/or CD138 and/or in which at least one of CD3, CD4, CD8, CD10, CD13, CD19, CD20, CD38, CD49d, CD58 and CD80 lacks.

13. The method according to claim 11 or 12, wherein an antibody and/or ligand are used for extracorporeal removal, which bind at least one or more of the group of CD26, CD29, CD44, CD54 and CD138.

14. The method according claim 1, wherein the disease is a cutaneous lymphoma and/or that the cell sample is a skin sample and/or that the at least one CMP includes one or more of the group of HLA-DQ, CD2, CD3, CD4, CD7, CD8, CD10, CD13, CD18, CD18, CD26, CD29, CD36, CD44, CD45, CD49f, CD54, CD56, CD57, CD58, CD62L, CD71, CD80 and HLA-DR and/or that the components are extracorporeally removed by means of at least one antibody and/or ligand, which bind at least one of the group of HLA-DQ, CD2, CD3, CD4, CD7, CD8, CD10, CD13, CD18, CD18, CD26, CD29, CD36, CD44, CD45, CD49f, CD54, CD56, CD57, CD58, CD62L, CD71, CD80 and HLA-DR.

15. The method according to claim 1, wherein the cell sample is again returned to the patient after the partial or complete extracorporeal removal of the components, which have the at least one determined CMP.

16. A device, which is formed for performing a method according to claim 1.

17. The device according to claim 16, which is formed for performing at least one apheresis method from the group of unselective apheresis, selective apheresis, whole-blood apheresis and photopheresis.

18. (canceled)
